(19) United States
(12) Patent Application Publication
(10) Pub. No.: US 2023/0049415 A1
(43) Pub. Date: Feb. 16, 2023

(54) CANNABINOIDS FOR USE IN TREATMENT

(71) Applicant: CAN-FITE BIOPHARMA LTD., PETACH TIKVA (IL)

(72) Inventors: Pnina FISHMAN, Herzliya (IL); Inbal ITZHAK, Kfar Saba (IL)

(73) Assignee: CAN-FITE BIOPHARMA LTD., PETACH TIKVA (IL)

(21) Appl. No.: 17/758,920

(22) PCT Filed: Jan. 14, 2021
(86) PCT No.: PCT/IL2021/050046

§ 371 Date: Jul. 15, 2022

(30) Foreign Application Priority Data
Jan. 16, 2020 (IL) 272078

(51) Int. Cl. A61K 36/185; A61K 31/352; A61K 31/05; A61K 31/60

(52) U.S. Cl. CPC A61K 36/185; A61K 31/352; A61K 31/05; A61K 31/60

(57) ABSTRACT

Cannabinoids are used to treat diseases or disorders treatable by an activator of the A₃ adenosine receptor (A₃AR). The cannabinoid effect is exerted through the A3AR. The cannabinoids may be selected from natural, phytocannabinoids, synthetic derivatives of phytocannabinoids, extracts of cannabis plant, and others.

### TECHNOLOGICAL FIELD

The present disclosure relates to medical uses of cannabinoids.

### BACKGROUND ART

References considered to be relevant as background to the presently disclosed subject matter are listed below:
U.S. Pat. No. 6,790,839 entitled “Pharmaceutical administration of adenosine agonists”
U.S. Pat. No. 7,141,553 entitled “A3AR agonists for the treatment of inflammatory arthritis”
U.S. Pat. No. 7,589,075 entitled “Use of an adenosine A3 receptor agonist for inhibition of viral replication”
U.S. Pat. No. 8,987,228 entitled “Pharmaceutical composition including an A3 adenosine receptor agonist . . . for treatment of psoriasis”
WO 2007/063538 entitled “Use of a3 adenosine receptor agonist in osteoarthritis treatment”
WO 2009/050707 entitled “Method for inducing hepatocyte proliferation and uses thereof”
WO 2017/090036 entitled “An a3 adenosine receptor ligand for use in treating ectopic fat accumulation”
WO 2013/111132 entitled “Treatment of liver conditions”
WO 2009/093018 entitled “ 7-hydroxy cannabidiol (7-oh-cbd) for use in the treatment of non-alcoholic fatty liver disease (nafld)”

Acknowledgement of the above references herein is not to be inferred as meaning that these are in any way relevant to the patentability of the presently disclosed subject matter.

### BACKGROUND

The Gi protein associated cell surface, the A3 adenosine receptor (A₃AR), has been demonstrated as an effective target for treatment of a variety of diseases or disorders. Furthermore, it has also been described that the A₃AR is over-expressed in cancer cells as well as in inflammatory cells and in peripheral blood mononuclear cells

(PBMCs) derived from patients with various auto-immune inflammatory diseases, such as rheumatoid arthritis psoriasis and Crohn's Disease.

Activation of the A₃AR with highly specific ligands, such as the A₃AR agonists 3-iodobenzyl-5′-N-methylc arboxamido adeno sine (piclidenososn) and 2-Chloro-N⁶-(3-iodobenzyl)-adenosine-5′-N-methyluronamide (namodenoson) was found to be effective in the treatment of cancer (U.S. Pat. No. 6,790,839 and WO 2013/111132), inflammatory diseases (U.S. Pat. Nos. 7,141,553, 8,987,228, WO 2007/063538), inhibiting viral replication (U.S. Pat. No. 7,589,075), inducing hepatocyte proliferation (WO 2009/050707), reducing ectopic fat accumulation (WO 2017/090036) and others.

Cannabinoids have also been described for their potential pharmaceutical use in the treatment of a variety of human diseases and disorders. An examples is the treatment of non-alcoholic fatty liver disease (NAFLD) by the use of 7-Hydroxy cannabidiol (7-OH-CBD), a metabolite of CBD (WO 2009/093018.

### GENERAL DESCRIPTION

The present disclosure provides, in accordance with a first of its aspects a formulation for treating a disease or disorder, that is treatable by an activator of the A₃ adenosine receptor (A₃AR) and that comprises at least one cannabinoid at an amount effective to treat or ameliorate the disease or disorder.

Provided by another aspect of this disclosure is a cannabinoid for treating a disease or disorder treatable by an A3AR activator.

Also provided by a further aspect of this disclosure is a method of treating a disease or disorder, treatable by and A3AR activator, comprising administering to a subject having said disease or disorder at least one cannabinoid in an amount effective to improve said disease or disorder or a condition exhibited by the subject as a result of said disease or disorder.

Also provided by an additional aspect of this disclosure is a method of treating a disease or disorder in a subject, comprising obtaining data on level of expression A3AR in tissue or cells of the subject; and when said data is indicative that expression level is elevated as compared to level of expression of the A3AR on cells or tissue of same lineage in subjects not suffering from said disease or condition, administering to the subject at least one cannabinoid in an amount effective to achieve improvement in said disease or condition.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C show the effect of cannabinoid extracts, comprising CBD/THC at nanomolar concentrations, on the proliferation of Stellate cells (FIG. 1A) which is reversed by an A₃AR antagonist (FIG. 1B) even when the CBD/THC formulation is given at pM concentrations (FIG. 1C)

FIG. 2 shows the effect of cannabinoid extract, comprising CBD/THC at nanomolar concentrations, in inhibiting proliferation of Human Hep-3b hepatocellular carcinoma cells.

FIG. 3 shows the effect of cannabinoid extract, comprising CBD/THC at nanomolar concentrations, on level of expression of various markers along the Wnt pathway, including and specifically, the level of the A₃ Adenosine receptor.

### DETAILED DESCRIPTION

Among the findings of the present disclosure is that the proliferation of Stellate cells is significantly inhibited when exposed to nM or even pM concentrations of a cannabinoid extract comprising Cannabidiol (CBD) and Tetrahydrocannabinol (THC). A similar inhibitory effect was exhibited when hepatocellular carcinoma cells were exposed to the same cannabinoid extract, an effect that was reversed when the same cells were exposed to an A₃ adenosine receptor (A₃AR) antagonist. These surprising findings suggest that the cannabinoid extract exerts this effect, at least partially, through the A₃AR.

Accordingly, cannabinoids, be it a single cannabinoid or a mixture of cannabinoids, may be used, according to this disclosure, for treating diseases, disorders or conditions that are treatable by an A₃AR activator (an A₃AR agonist or allosteric modulator). The term “clinical condition” will be used hereinafter to collectively denote said disease, disorder, or condition. The terms “treatment”, “treating” or “treat” is being used herein to refer to the therapeutic administration of cannabinoids to subjects having said clinical condition, in accordance with this disclosure. A specific subset of such treatment is of subjects in which the clinical condition is associated with an elevated expression the A₃AR as compared to the expression of the A₃AR in healthy subjects (i.e., that are not diagnosed as having said clinical condition).

The term treatable denotes, among others, that: (i) said clinical condition, the physiological manifestation thereof, or symptoms associated therewith may be controlled (typically reduced) by the administration of an A₃AR activator, such as an agonist of A₃AR such as piclidenoson or namodenoson (also known in the scientific literature as IB-MECA and Cl-IB-MECA, respectively); (ii) following such administration an improvement in general health scores (by a physician assessment or by a patient's own assessment, e.g., an improvement in the ACR score in rheumatoid arthritis patients, PASI or PGA scores in psoriasis and many others); or (iii) that such administration yields an improvement in the treated subject's quality of life. The cannabinoid is used, in accordance with this disclosure, for the treatment of a clinical condition otherwise treatable with an A₃AR activator, the treatment by the cannabinoid being in the alternative or in addition to the A₃AR activator.

Accordingly, the at least one cannabinoid acts, in accordance with this disclosure, as an A₃AR activator.

Specifically, in accordance with the present disclosure there is provided a formulation comprising at least one cannabinoid for use in treating said clinical condition; a cannabinoid for use in such treatment; and a method for said treatment.

In the following, when referring to elements of the formulation, it is to be understood as equally defining elements of the use or method disclosed herein, and vice versa, the description relating to the method disclosed herein should be regarded as equally, and mutatis mutandis, defining the formulation disclosed herein.

The formulation used in the context of the present disclosure comprises at least one cannabinoid. The formulation may comprise a cannabis oil, cannabis concentrate, cannabis extract, a natural isolated cannabinoid, any chemical derivative of a natural cannabinoid or a synthetic cannabinoid. Phytocannabinoids, namely cannabinoids derived from a plant such as cannabis plant are a specific example of cannabinoids used in accordance with this disclosure. There are at least 113 different known phyotcannabinoids, including THC (tetrahydrocannabinol) or CBD (cannabidiol) and their derivatives.

The cannabinoids can be chemically classified into distinct chemical classes: the classical cannabinoids that are structurally related to THC or CBD; the nonclassical cannabinoids (cannabimimetics) including the aminoalkylindoles, 1,5-diarylpyrazoles, quinolines, and arylsulfonamides; and others.

Non-limiting examples of phytocannabinoids that may be potentially used in accordance with the present disclosure includes selected from the group consisting of Tetrahydrocannabinolic Acid (THCA), Tetrahydrocannabinol (THC), Cannabidiolic Acid (CBDA), Cannabidiol (CBD), Cannabinol (CBN), Cannabigerol (CBG), Cannabichromene (CBC), Cannabicyclol (CBL), Cannabivarin (CBV), Cannabichromevarin (CBCV), Cannabigerovarin (CBGV), Cannabigerol monomethyl ether (CBGM), Cannabielsoin (CBE), Cannabicitran (CBT), Tetrahydrocannabivarin (THCV) and Cannabidivarin (CBDV). Also contemplated for use according to this disclosure are synthetic derivatives of these phytocannabinoids. The cannabinoids may be selected on the basis of their A₃AR agonistic activity, as explained below.

In accordance with the present disclosure, the formulation can include a single or any combinations of n cannabinoids (n being an integer, such as 1, 2, 3, 4, 5, 6, 7, 8, 9, etc.) of the above non-limiting examples of cannabinoids.

In the context of the present disclosure, when referring to a phytocannabinoid, or a cannabinoid (one or more) derived from a plant, it is to be understood as being any one of a plant-derived material including plant extract, a plant concentrate, a plant isolate, a plant-derived oil and/or one or more cannabinoid compounds isolated from the plant material; the plant being typically (although not exclusively) a cannabis plant. CBD and THC, whether purified, synthetic or whether provided in the form of a plant-derived material, are examples of phytocannabinoids.

The formulation of this disclosure is used for the treatment of a clinical condition treatable by an A₃AR activator, for example by an A₃AR agonists such as piclidenoson or namodenoson. The selection of the cannabinoid to be used for treatment may be through studies conducted in the appropriate in vitro or animal disease models or through the conduct of the appropriate human clinical study. Cannabinoids may be screened in such studies for those having an effect that is similar to that achieved with the A₃AR activator used as the comparable in such studies. To ensure that the selected cannabinoid acts through the A₃AR, an antagonist of that receptor may be used to examine whether the effect exerted by the cannabinoid is reduced or even eliminated by said antagonist. By comparing effects with or without an antagonist to the A₃AR, a cannabinoid with an effect exerted specifically or a least primarily through the A₃AR may be selected.

By an embodiment of this disclosure the cannabinoid that is used is one that exerts it effect primarily through the A₃AR; particularly, have an A₃AR activator effect, meaning that it can bind to and activate this receptor. It should be noted, however, that while the at least one cannabinoid used in accordance with this embodiment exerts its effect primarily through an A₃AR activator effect, it is possible that the cannabinoid has a parallel effect, e.g., exerted through other receptors on the same or other cells. For example, the cannabinoid used in accordance with this embodiment may have an effect of reducing disease symptoms of the clinical condition, e.g., an anti-inflammatory or anti-cancer effect, that is exerted through the A₃AR receptor and have in parallel also an effect of reducing pain, an effect on the general well-being of the subject, or another general effect.

An A₃AR activator or A₃AR activator effect denotes an effect exerted directly on the A₃AR (e.g., via the adenosine binding site) or indirectly (e.g., via an allosteric binding site) to thereby activate the A₃AR, including full or partial activation of this receptor. The A₃AR activator effect is thus an enhancement of the activity of the A₃AR by (i) agonistic activation via the receptor's adenosine binding site to thereby induce a direct activation of the receptor, or (ii) allosteric modulation of the receptor via an allosteric binding site.

An A₃AR activator effect can, as noted above, be reduced or, at times, totally eliminated by an antagonist of this receptor. This is one of the typical characteristics of said effect and it may be tested in vitro with cells that express the A₃AR.

A subset of clinical conditions treatable by cannabinoids in accordance with this disclosure is one in which there is an elevated level of expression of an A₃AR in cells or tissue, as compared to cells or tissue of the same lineage in subjects not suffering from said or disorder. Such increased level may be an average expression level of the A₃AR in said cells or tissue in subjects suffering from said clinical condition, which is at least 1.5 times the average expression level of the A₃AR in cells or tissue of a same lineage in subjects not suffering from said clinical condition. While a clinical condition may be selected as a therapeutic target by such average increase in A₃AR expression level, the patients may also be selected individually as recipients of the cannabinoid-based treatment according to this disclosure, based on a pre-treatment testing of the A₃AR level and selecting only those subjects with an elevated expression level of this receptor versus that in subjects not suffering from said clinical condition. The elevated expression level of the A₃AR may be at least 1.5, 1.6, 1.7, 1.8, 1.9, or at least 2 times that of subjects not suffering from said clinical condition; but may also be, at times, at least 2.5 or at least 3 times that of subjects not suffering from said clinical condition.

By one embodiment the cells or tissue exhibiting the elevated A₃AR expression are cells or tissue of a diseased organ or tissue having a disease-related abnormality; e.g., cancer cells, inflammatory cells, cells of the immune system, adipose cells, liver cells. In some clinical conditions the elevated A₃AR expression may be on other cells, for example on circulating while blood cells, e.g., mononuclear cells.

Examples of the clinical condition treatable in accordance with this disclosure are cancer, inflammatory disease, liver disease such as NAFLD or obesity.

It has been found that the formulation comprising at least one cannabinoid is effective, in in vitro assays, at very low concentrations, even at the pM range. Thus, irrespective of the form of administration, and in accordance with some examples, the concentration of the at least one cannabinoid in the formulation is sufficient to induce, after administration, a peak blood concentration of the least one cannabinoid within the range of 0.01-100 nM, at times, within the range of 0.01-50 nM, at times, below 50 nM, at times, between 0.1 μM and 10 nM, at times, between 10 μM and 1 nM, at times between 1 pM and 20 nM or any range between 1 pM and 100 nM.

By an embodiment of this disclosure, the at least one cannabinoid is administered to the subject in combination with an A₃AR agonist, such as piclidenoson or namodenoson. Such combination means that the A₃AR agonist may be co-administered with the at least one cannabinoid, for example, within the same formulation, or may be administered to the subject within the same therapeutic regiment, for example one given once, twice or thrice daily and the other given once, twice or thrice daily at different times.

Oral administration of the at least one cannabinoid is one exemplary administration form. Parenteral is another. Accordingly, the at least one cannabinoid may be formulated in a dosage form suitable for, respective, oral and parenteral delivery. Other examples of delivery forms are by inhalation, oro-mucosal or sublingual administration, topical administration or rectal administration.

The therapeutic treatment according to this disclosure may be for a short time period of 1 day, several days or several weeks, or may be a chronic treatment over prolonged time periods of months to years. Chronic treatement it is to be understood as involving routine administration of the at least one cannabinoid for a prolonged time period (as opposed to a single time administration).

The clinical conditions treatable according to this disclosure include, for examples, inflammation, cancer, e.g., liver cancer, non-alcoholic fatty liver disease (NAFLD) or other liver conditions, obesity, fibrosis, e.g., liver fibrosis, neuropathic pain.

The A₃AR activator effect encompasses A₃AR agonistic effect or A₃AR allosteric modulator effect.

An A₃AR agonistic effect denotes an effect that is exerted through binding to the adenosine binding site of this receptor, thereby fully or partially activating the A₃ adenosine receptor.

When referring to A₃AR allosteric modulator effect denotes an effect that is exerted through binding at the receptor's allosteric site, which may be different from the binding site of the endogenous ligand or agonist thereof, to thereby impart a positive regulation on the receptor's activity. Such modulation may be (i) an increased affinity to binding of adenosine or an A₃AR agonists to the receptor's adenosine binding site (the orthosteric binding site) and/or (ii) a decrease in dissociation rate of adenosine or an A₃AR ligand to the orthosteric binding site.

The method disclosed herein may also involve, in accordance with the invention the step of obtaining information with respect to the expression level of the A₃AR in the tissue or cells indicative of the existence of the disease or condition to be treated, so as to determined that the subject is suitable for the treatment. Thus, prior to the administration of the at least one cannabinoid, the subject or the physician responsible for the subject's treatment is of knowledge/or in possession of data indicative of the level of expression of the A₃AR in tissue or cells of the subject and the subject is administered with the at least one cannabinoid only if the level of expression is above a predefined reference level, i.e., is considered by predetermined parameters to be elevated levels that require the treatment.

As used herein, the forms “a”, “an” and “the” include singular as well as plural references unless the context clearly dictates otherwise. For example, the term “a cannabinoid” includes one or more cannabinoids.

Further, as used herein, the term “comprising” is intended to mean that the composition include the recited active agent, i.e. a cannabinoid, but not excluding other elements, such as physiologically acceptable carriers and excipients as well as other active agents.

Further, all numerical values, e.g. when referring the amounts or ranges of the elements constituting the formulations comprising the at least one cannabinoid as an active ingredient, are approximations which are varied (+) or (−) by up to 20%, at times by up to 10% of from the stated values. It is to be understood, even if not always explicitly stated that all numerical designations are preceded by the term “about”.

### Embodiments

Defined in the following numbered paragraphs are embodiments of the disclosure herein. These embodiments are intended to add and not limit the above disclosure.
1. A formulation for treating a disease or disorder treatable by an agonist of the A3 adenosine receptor (A₃AR), comprising an amount of at least one cannabinoid effective to treat or ameliorate said disease or disorder.
2. The formulation of embodiment 1, wherein said cannabinoid exerts it effect primarily through the A₃AR.
3. The formulation of embodiment 1 or 2, wherein said cannabinoid has a physiological effect or therapeutic effect on said disease or disorder that is reduced by an antagonist of the A₃AR.
4. The formulation of any one of embodiments 1 to 3, wherein the effect exerted on a target cell is reduced by an antagonist of the A₃AR.
5. The formulation of any one of embodiments 1 to 4, wherein said cannabinoid exerts a physiological effect on cells in vitro that is reduced by an antagonist of the A₃AR.
6. The formulation of any one of embodiments 1 to 5, wherein the at least one cannabinoid has an A₃AR activator effect.
7. The formulation of embodiment 6, wherein the A₃AR activator effect is exerted through the orthosteric or allosteric binding site of the receptor.
8. The formulation of embodiment 7, wherein the at least one cannabinoid has an A₃AR agonistic activity.
9. The formulation of any one of embodiments 1 to 8, wherein the disease or disorder is associated with an elevated level of expression of an A₃AR in cells or tissue, as compared to cells or tissue of the same lineage in subjects not suffering from said or disorder.
10. The formulation of embodiment 9, wherein said elevated level is an average expression level of the A₃AR in said cells or tissue in subjects suffering from said disease or condition, which is at least 1.5 times the average expression level of the A₃AR in cells or tissue of a same lineage in subjects not suffering from said disease or condition.
11. The formulation of embodiment 10 or 11, wherein the cells or tissue exhibiting said elevated expression are cells or tissue of a diseased organ or tissue having a disease-related abnormality.
12. The formulation of embodiment 11, wherein the diseased cells are cancer cells, inflammatory cells, cells of the immune system, adipose cells, liver cells.
13. The formulation of any one of embodiments 1 to 12, wherein the disease or disorder is cancer, inflammatory disease, liver disease such as NAFLD or obesity.
14. The formulation of any one of embodiments 1 to 13, comprising at least one cannabinoid selected from the group consisting of (i) tetrahydrocannabinolic acid (THCA), tetrahydrocannabinol (THC), cannabidiolic acid (CBDA), cannabidiol (CBD), cannabinol (CBN), cannabigerol (CBG), cannabichromene (CBC), tetrahydrocannabivarin (THCV), cannabidivarin (CBDV); and (ii) chemical derivatives of the cannabinoids of (i).
15. The formulation of any one of embodiments 1 to 14, comprising an extract of cannabis plant.
16. The formulation of any one of embodiments 1 to 15, wherein said at least one cannabinoid is in an amount sufficient to induce, after administration, a blood concentration of said least one cannabinoid within the range of 0.01-100 nanomolar.
17. The formulation of any one of embodiments 1 to 16, wherein said at least one cannabinoid is in an amount sufficient to induce a blood concentration of said least one cannabinoid of 0.01 to 1 nanomolar.
18. The formulation of one of embodiments 1 to 17, comprising also an A₃AR activator, e.g., binding to and activating the receptor through the orthosteric or allosteric binding site of the receptor.
19. The formulation of embodiment 18 wherein the A₃AR activator is an A₃AR agonist, e.g., piclidenoson or namodenoson.
20. The formulation of any one of embodiments 1 to 19, in a dosage form suitable for oral delivery.
21. A cannabinoid for treating a disease or disorder treatable by an agonist of the A3 adenosine receptor (A₃AR).
22. The cannabinoid of embodiment 21, exerting it effect primarily through the A₃AR.
23. The cannabinoid of embodiment 21 or 22, having a physiological effect or therapeutic effect on said disease or disorder that is reduced by an antagonist of the A₃AR.
24. The cannabinoid of any one of embodiments 21 to 23, having an effect on a target cell that is reduced by an antagonist of the A₃AR.
25. The cannabinoid of any one of embodiments 21 to 24, having a physiological effect on cells in vitro that is reduced by an antagonist of the A₃AR.
26. The cannabinoid of any one of embodiments 21 to 25, having an A₃AR activator effect.
27. The cannabinoid of embodiment 26, wherein the A₃AR activator effect is exerted through the orthosteric or allosteric binding site of the receptor.
28. The cannabinoid of embodiment 27, having an A₃AR agonistic activity.
29. The cannabinoid of any one of embodiments 21 to 28, wherein the disease or disorder is associated with an elevated level of expression of an A₃AR in cells or tissue, as compared to cells or tissue of the same lineage in subjects not suffering from said or disorder.
30. The cannabinoid of embodiment 29, wherein said elevated level is an average expression level of the A₃AR in said cells or tissue in subjects suffering from said disease or condition, which is at least 1.5 times the average expression level of the A₃AR in cells or tissue of a same lineage in subjects not suffering from said disease or condition.
31. The cannabinoid of embodiment 29 or 30, wherein the cells or tissue exhibiting said elevated expression are cells or tissue of a diseased organ or tissue having a disease-related abnormality.
32. The cannabinoid of embodiment 31 wherein the diseased cells are cancer cells, inflammatory cells, cells of the immune system, adipose cells, liver cells.
33. The cannabinoid of any one of embodiments 21 to 32, wherein the disease or disorder is cancer, inflammatory disease, liver disease such as NAFLD or obesity.
34. The cannabinoid of any one of embodiments 21 to 33, comprising at least one cannabinoid selected from the group consisting of (i) tetrahydrocannabinolic acid (THCA), tetrahydrocannabinol (THC), cannabidiolic acid (CBDA), cannabidiol (CBD), cannabinol (CBN), cannabigerol (CBG), cannabichromene (CBC), tetrahydrocannabivarin (THCV), cannabidivarin (CBDV); and (ii) chemical derivatives of the cannabinoids of (i).
35. The cannabinoid of any one of embodiments 21 to 34, form treating in combination with an extract of cannabis plant.
36. The cannabinoid of any one of embodiments 21 to 35, in an amount sufficient to induce, after administration, a blood concentration of said least one cannabinoid within the range of 0.01-100 nanomolar.
37. The cannabinoid of any one of embodiments 21 to 36, in an amount sufficient to induce a blood concentration of said least one cannabinoid of 0.01 to 1 nanomolar.
38. The cannabinoid of one of embodiments 21 to 37, exerting an effect of an A₃AR activator, e.g., binding to and activating the receptor through the orthosteric or allosteric binding site of the receptor.
39. The cannabinoid of embodiment 38, wherein the A₃AR activator is an A₃AR agonist, e.g., piclidenoson or namodenoson.
40. The cannabinoid of any one of embodiments 18 to 33, in a dosage form suitable for oral delivery.
41. A method of treating a disease or disorder treatable by an agonist of the A3 adenosine receptor (A₃AR), comprising administering to a subject having said disease or condition at least one cannabinoid in an amount effective to improve said disease or disorder or a condition exhibited by the subject as a result of said disease or disorder.
42. The method of embodiment 35, wherein said cannabinoid exerts it effect primarily through the A₃AR.
43. The method of embodiment 35 or 36, wherein said cannabinoid has a physiological effect or therapeutic effect on said disease or disorder that is reduced by an antagonist of the A₃AR.
44. The method of any one of embodiments 35 to 37, wherein the effect exerted on a target cell is reduced by an antagonist of the A₃AR.
45. The method of any one of embodiments 35 to 38, wherein said cannabinoid exerts a physiological effect on cells in vitro that is reduced by an antagonist of the A₃AR.
46. The method of any one of embodiments 41 to 45, wherein the at least one cannabinoid has an A₃AR activator effect.
47. The formulation of embodiment 46, wherein the A₃AR activator effect is exerted through the orthosteric or allosteric binding site of the receptor.
48. The formulation of embodiment 47, wherein the at least one cannabinoid has an A3AR agonistic activity.
49. The method of any one of embodiments 41 to 48, wherein the disease or disorder is associated with an elevated level of expression of an A₃AR in cells or tissue, as compared to cells or tissue of the same lineage in subjects not suffering from said or disorder.
50. The method of embodiment 49, wherein said elevated level is an average expression level of the A₃AR in said cells or tissue in subjects suffering from said disease or condition, which is at least 1.5 times the average expression level of the A₃AR in cells or tissue of a same lineage in subjects not suffering from said disease or condition.
51. The method of embodiment 41 or 50, wherein the cells or tissue exhibiting said elevated expression are cells or tissue of a diseased organ or tissue having a disease-related abnormality.
52. The method of embodiment 51, wherein the diseased cells are cancer cells, inflammatory cells, cells of the immune system, adipose cells, liver cells.
53. The method of any one of embodiments 41 to 52, wherein the disease or disorder is cancer, inflammatory disease, liver disease such as NAFLD or obesity.
54. The method of any one of embodiments 41 to 53, comprising at least one cannabinoid selected from the group consisting of (i) tetrahydrocannabinolic acid (THCA), tetrahydrocannabinol (THC), cannabidiolic acid (CBDA), cannabidiol (CBD), cannabinol (CBN), cannabigerol (CBG), cannabichromene (CBC), tetrahydrocannabivarin (THCV), cannabidivarin (CBDV); and (ii) chemical derivatives of the cannabinoids of (i).
55. The method of any one of embodiments 41 to 54, comprising an extract of cannabis plant.
56. The method of any one of embodiments 41 to 55, wherein said at least one cannabinoid is in an amount sufficient to induce, after administration, a blood concentration of said least one cannabinoid within the range of 0.01-100 nanomolar.
57. The method of any one of embodiments 41 to 56, wherein said at least one cannabinoid is in an amount sufficient to induce a blood concentration of said least one cannabinoid of 0.01 to 1 nanomolar.
58. The method of one of embodiments 41 to 57, comprising also administering to the subject an A₃AR activator that binds to and activates the receptor through the orthosteric or allosteric binding site of the receptor.
59. The method of embodiment 58, wherein the A₃AR activator is an A₃AR agonist, e.g., piclidenoson or namodenoson.
60. The method of any one of embodiments 41 to 59, wherein said cannabinoid is administered orally.
61. A method of treating a disease or disorder in a subject, comprising obtaining data on level of expression A₃AR in tissue or cells of the subject; and when said data is indicative that expression level is elevated as compared to level of expression of the A₃AR on cells or tissue of same lineage in subjects not suffering from said disease or condition, administering to the subject at least one cannabinoid in an amount effective to achieve improvement in said disease or condition.
62. The method of embodiment 61, wherein said cannabinoid exerts it effect primarily through the A₃AR.
63. The method of embodiment 61 or 62, wherein said cannabinoid has a physiological effect or therapeutic effect on said disease or disorder that is reduced by an antagonist of the A₃AR.
64. The method of any one of embodiments 61 to 54, wherein the effect exerted on a target cell is reduced by an antagonist of the A₃AR.
65. The method of any one of embodiments 61 to 64, wherein said cannabinoid exerts a physiological effect on cells in vitro that is reduced by an antagonist of the A₃AR.
66. The method of any one of embodiments 61 to 65, wherein the at least one cannabinoid has an A₃AR activator effect.
67. The formulation of embodiment 66, wherein the A₃AR activator effect is exerted through the orthosteric or allosteric binding site of the receptor.
68. The formulation of embodiment 67, wherein the at least one cannabinoid has an A₃AR agonistic activity.
69. The method of any one of embodiments 61 to 68, wherein said elevated level is an average expression level of the A₃AR in said cells or tissue in subjects suffering from said disease or condition, which is at least 1.5 times the average expression level of the A₃AR in cells or tissue of a same lineage in subjects not suffering from said disease or condition.
70. The method of any one of embodiments 61 to 69, wherein the cells or tissue exhibiting said elevated expression are cells or tissue of a diseased organ or tissue having a disease-related abnormality.
71. The method of embodiment 70, wherein the diseased cells are cancer cells, inflammatory cells, cells of the immune system, adipose cells, liver cells.
72. The method of any one of embodiments 61 to 71, wherein the disease or disorder is cancer, inflammatory disease, liver disease such as NAFLD or obesity.
73. The method of any one of embodiments 61 to 72, comprising at least one cannabinoid selected from the group consisting of (i) tetrahydrocannabinolic acid (THCA), tetrahydrocannabinol (THC), cannabidiolic acid (CBDA), cannabidiol (CBD), cannabinol (CBN), cannabigerol (CBG), cannabichromene (CBC), tetrahydrocannabivarin (THCV), cannabidivarin (CBDV); and (ii) chemical derivatives of the cannabinoids of (i).
74. The method of any one of embodiments 61 to 73, comprising an extract of cannabis plant.
75. The method of any one of embodiments 61 to 74, wherein said at least one cannabinoid is in an amount sufficient to induce, after administration, a blood concentration of said least one cannabinoid within the range of 0.01-100 nanomolar.
76. The method of any one of embodiments 61 to 75, wherein said at least one cannabinoid is in an amount sufficient to induce a blood concentration of said least one cannabinoid of 0.01 to 1 nanomolar.
77. The method of one of embodiments 61 to 76, comprising also administering to the subject an A₃AR activator that binds to and activates the receptor through the orthosteric or allosteric binding site of the receptor.
78. The method of embodiment 77, wherein the A₃AR activator is an A₃AR agonist, e.g., piclidenoson or namodenoson.
79. The method of any one of embodiments 61 to 78, wherein said cannabinoid is administered orally.

### DETAILED DESCRIPTION OF NON-LIMITING EXAMPLES

The disclosure will now be exemplified in the following description of experiments that were carried out in accordance with the teaching herein. It is to be understood that these examples are intended to be in the nature of illustration rather than of limitation. Obviously, many modifications and variations of these examples are possible in light of the above teaching.

Inhibition of Human Hepatocellular Carcinoma and Human Hepatic Stellate Cell Growth by CBD/THC extract

### Materials & Methods

Cell culture. Hep-3B hepatocellular carcinoma cells and LX-2 hepatic stellate cells were grown in MeM-Eagle medium. All media contained penicillin (10 units/ml), streptomycin (10 μg/ml), L-glutamine (2 mM) and 10% fetal bovine serum (FBS). The cells were maintained in T-75 flasks at 37° C. in a 5% CO₂ incubator and transferred to a freshly prepared medium twice weekly. For all studies serum starved cells were used. FBS was omitted from the cultures for 18 hours and the experiment was carried out on monolayers of cells in DMEM medium supplemented with 1% FBS in a 37° C., 5% in a CO₂ incubator. CBD/THC in different ratios (C15/T3 and C3/T15) was introduced to the culture system at concentrations of 100 pM, 1, 10 and 100 nM (stock solution of 100 mM at DMSO was prepared from the original oil and further diluted in culture medium to reach the nM concentration in the experimental system.

³H-thymidine incorporation assay. ³H-thymidine incorporation assay was used to evaluate cell growth. cells (5,000 cells/well) were incubated with different concentration of T3/C15, T15/C3 in 96-well plate for 48/72 hours. Each well was pulsed with 1 μCi ³H-thymidine for the last 24 hours. Cells were harvested and the ³H-thymidine uptake was determined in an LKB liquid scintillation counter (LKB, Piscataway, N.J., USA). These experiments were repeated at least 4 times.

Western Blot Analysis. Protein extract from cell line were utilized. The samples were homogenized with an ice-cold RIPA lysis buffer (Thermo scientific; 89900) with protease phosphatase inhibitor cocktail (Thermo scientific; 1861281). Cell debris were removed by centrifugation for 10 min, at 7500 g. The supernatant was utilized for Western Blot (WB) analysis. Protein concentrations were determined using the NanoDrop (ThermoFisher Scientific, Mass., US). Equal amounts of the sample (50 μg) were separated by SDS -PAGE, using 4-12% polyacrylamide gels. The resolved proteins were then electro blotted onto nitrocellulose membranes (Pall Corporation, Fla., US). Membranes were blocked with 5% bovine serum albumin and incubated with the desired primary antibody (Santa cruz; A3AR sc-13938, PI3K sc-1637,GSK-3β sc-9166, β-catenin sc-7963, cyclin D1 sc-8396, NF-κB sc-372, LEF-1 sc-374522, α-SMA sc-32251 and β-actin sc-47778 dilution 1:1000) for 24 h hour at 4° C. Blots were then washed and incubated with a secondary antibody (Abcam; Mouse ab97020, Rabbit ab97048) for 1 h at room temperature. Bands were recorded using BCIP/NBT color development kit (Promega, Madison, Wis., USA). Densitometry of protein expression was normalized against β-actin and expressed as % of control.

### Results

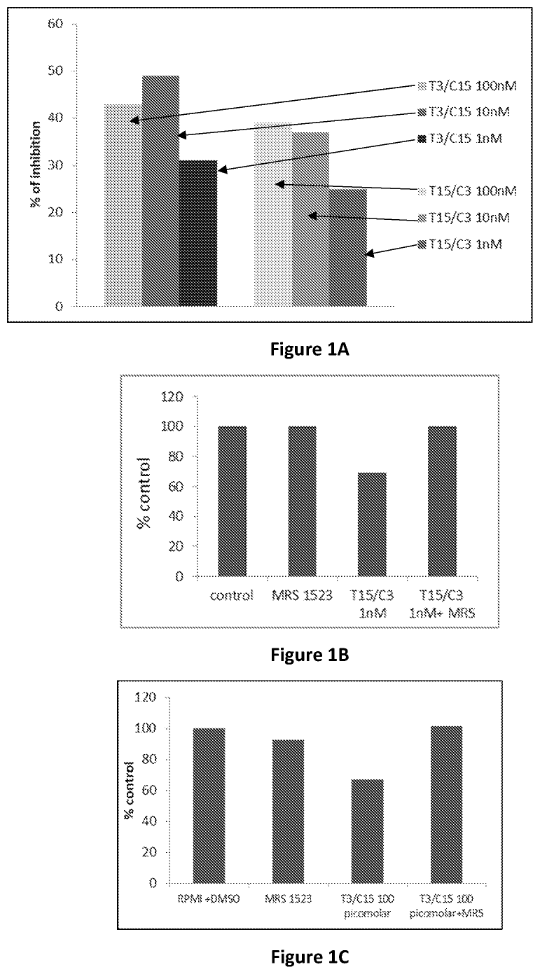

FIG. 1A shows that the CBD/THC containing extract was effective in inhibiting the proliferation of Stellate cells at nanomolar concentrations, irrespective of the ratio between the two cannabinoids.

FIGS. 1B and 1C show that the inhibitory effect of the CBD/THC containing extract (at pM and nM concentrations) is neutralized when an A₃AR antagonist MRS1523 is added, thus teaching that the effect of the extract acts as an agonist.

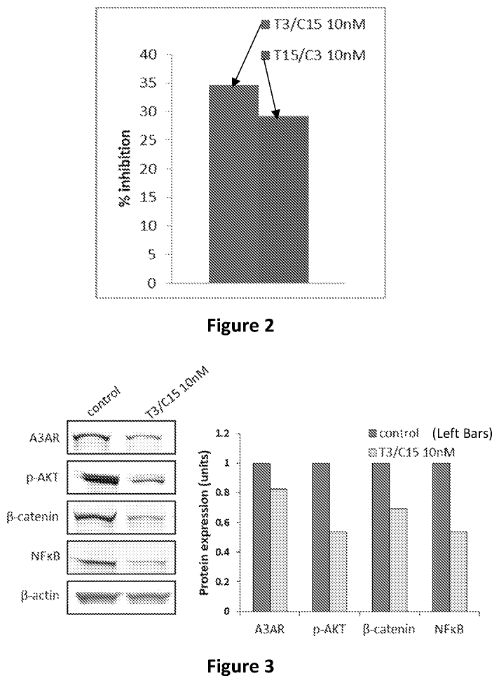

In addition, FIG. 2 shows that cannabinoid extracts containing CBD/THC at different ratios (C15/T3 and C3/T15) was effective in inhibiting proliferation of Human Hep-3b hepatocellular carcinoma cells at 10 nM concentration.

FIG. 3 shows that the CBD/THC containing extracts and specifically C15/C3 decrease the level of A₃ Adenosine receptor, thus demonstrating that C15/T3 induced an anti-proliferative effect via the A₃AR. Additional cell signaling molecules were also down-regulated upon treatment with the C15/T3 and entailed pAKT, NF- [symbol] B and β-catenin, showing the involvement of both the NF- [symbol] B and the Wnt/β-catenin pathways in mediating the anti-cancer and the liver anti-fibrotic effects of the cannabinoids.

1.-39. (canceled)

40. A formulation for treating a disease or disorder treatable by an agonist of the A3 adenosine receptor (A₃AR), comprising an amount of at least one cannabinoid effective to treat or ameliorate said disease or disorder, wherein said cannabinoid exerts it effect primarily through the A₃AR.

41. The formulation of claim 40, wherein said cannabinoid has a physiological effect or therapeutic effect on said disease or disorder that is reduced by an antagonist of the A₃AR.

42. The formulation of claim 40, wherein the at least one cannabinoid has an A₃AR activator effect or an A₃AR agonistic activity.

43. The formulation of claim 40, wherein the disease or disorder is associated with an elevated level of expression of an A₃AR in cells or tissue, as compared to cells or tissue of the same lineage in subjects not suffering from said or disorder.

44. The formulation of claim 40, comprising at least one cannabinoid selected from the group consisting of: (i) tetrahydrocannabinolic acid (THCA), tetrahydrocannabinol (THC), cannabidiolic acid (CBDA), cannabidiol (CBD), cannabinol (CBN), cannabigerol (CBG), cannabichromene (CBC), tetrahydrocannabivarin (THCV), cannabidivarin (CBDV); (ii) chemical derivatives of the cannabinoids of (i); and (iii) an extract of cannabis plant.

45. The formulation of claim 40, comprising also an activator of the A₃AR.

46. The formulation of claim 45, wherein said activator is an A₃AR agonist.

47. A method of treating a disease or disorder treatable by an agonist of the A3 adenosine receptor (A₃AR), comprising administering to a subject having said disease or condition at least one cannabinoid in an amount effective to improve said disease or disorder or a condition exhibited by the subject as a result of said disease or disorder, wherein said cannabinoid exerts it effect primarily through the A₃AR.

48. The method of claim 47, wherein said cannabinoid has a physiological effect or therapeutic effect on said disease or disorder that is reduced by an antagonist of the A₃AR.

49. The method of claim 47, wherein the at least one cannabinoid has an A₃AR activator effect or an A₃AR agonistic activity.

50. The method of claim 47, wherein the disease or disorder is associated with an elevated level of expression of an A₃AR in cells or tissue, as compared to cells or tissue of the same lineage in subjects not suffering from said or disorder.

51. The method of claim 47, comprising administering to the subject at least one cannabinoid selected from the group consisting of: (i) tetrahydrocannabinolic acid (THCA), tetrahydrocannabinol (THC), cannabidiolic acid (CBDA), cannabidiol (CBD), cannabinol (CBN), cannabigerol (CBG), cannabichromene (CBC), tetrahydrocannabivarin (THCV), cannabidivarin (CBDV); (ii) chemical derivatives of the cannabinoids of (i); and (iii) an extract of cannabis plant.

52. The method of claim 47, comprising also an activator of the A₃AR.

53. The method of claim 52, wherein said activator is an A₃AR agonist.

54. A method of treating a disease or disorder in a subject, comprising obtaining data on level of expression A₃AR in tissue or cells of the subject; and when said data is indicative that expression level is elevated as compared to level of expression of the A₃AR on cells or tissue of same lineage in subjects not suffering from said disease or condition, administering to the subject at least one cannabinoid in an amount effective to achieve improvement in said disease or condition, wherein said cannabinoid exerts it effect primarily through the A₃AR.

55. The method of claim 54, wherein said cannabinoid has a physiological effect or therapeutic effect on said disease or disorder that is reduced by an antagonist of the A₃AR.

56. The method of claim 54, wherein the at least one cannabinoid has an A₃AR activator effect or an A₃AR agonistic activity.

57. The method of claim 54, wherein the disease or disorder is associated with an elevated level of expression of an A₃AR in cells or tissue, as compared to cells or tissue of the same lineage in subjects not suffering from said or disorder.

58. The method of claim 54, comprising administering to the subject at least one cannabinoid selected from the group consisting of: (i) tetrahydrocannabinolic acid (THCA), tetrahydrocannabinol (THC), cannabidiolic acid (CBDA), cannabidiol (CBD), cannabinol (CBN), cannabigerol (CBG), cannabichromene (CBC), tetrahydrocannabivarin (THCV), cannabidivarin (CBDV); (ii) chemical derivatives of the cannabinoids of (i); and (iii) an extract of cannabis plant.

59. The method of claim 54, comprising also an activator of the A₃AR.
